(19) United States
(12) Patent Application Publication
(10) Pub. No.: US 2021/0260203 A1
(43) Pub. Date: Aug. 26, 2021

(54) POLYPEPT(O)ID-BASED GRAFT COPOLYMERS FOR IN VIVO IMAGING BY TETRAZINE TRANSYCLOOCTENE CLICK CHEMISTRY

(71) Applicants: RIGSHOSPITALET, Copenhagen Ø (DK); JOHANNES GUTENBERG UNIVERSITY, Mainz (DE); UNIVERSITY OF COPENHAGEN, Copenhagen K (DK)

(72) Inventors: Andreas KJÆR, Frederiksberg (DK); Matthias Manfred HERTH, Malmoe (SE); Matthias BARZ, Worms (DE); Alexander Oksar BIRKE, Mainz (DE)

(21) Appl. No.: 17/256,309

(22) PCT Filed: Jun. 26, 2019
(86) PCT No.: PCT/EP2019/067060

§ 371 Date: Dec. 28, 2020

(30) Foreign Application Priority Data
Jun. 29, 2018 (DK) PA 2018 70453

(51) Int. Cl. A61K 47/64; A61K 47/66; A61K 51/08; A61K 51/12; A61K 51/04

(52) U.S. Cl. CPC A61K 47/645; A61K 47/66; B82Y 5/00; A61K 51/1244; A61K 51/0495; A61K 51/088

(57) ABSTRACT

There is provided novel polypeptide-based carrier systems, which make it possible to label polymeric nanoparticles in the living organism. This enables new approaches in tumor diagnostics (high signal to background ratio) and radiotherapy (radiotherapy of solid tumors). The polypeptide-based carrier system comprises a polypept(o)idic comb (graft) copolymer, and one or more tetrazine bioorthogonal functional groups each linked to a diagnostic agent

### FIELD OF THE INVENTION

The present invention relates to polypeptide-based carrier systems, which make it possible to label polypept(o)ide based brush copolymers in the living organism by tetrazine transcyclooctene ligation.

### BACKGROUND OF THE INVENTION

Active targeting of an organ or a tissue is achieved by the direct or indirect conjugation of the desired active moieties (e.g. a contrast enhancing agent or a cytotoxic compound) to a targeting construct, which binds to cell surfaces or promotes cellular uptake at or near the target site of interest. The targeting moieties used to target such agents are typically constructs that have affinity for cell surface targets (e.g., membrane receptors), structural proteins (e.g., amyloid plaques), or intracellular targets (e.g., RNA, DNA, enzymes, cell signaling pathways). These moieties can be antibodies (fragments), proteins, aptamers, oligopeptides, oligonucleotides, oligosaccharides, as well as peptides, peptoids and organic drug compounds known to accumulate at a particular disease or malfunction. In addition, passive targeting can occur for long circulating nanoparticles above 10 nm in hydrodynamic diameter. These particles can only extravasate from the blood stream in tissues where the vasculature is leaky, which can be the case in inflamed or cancerous tissues. Therefore, long circulating nanoparticles can provide a certain ability for targeting of solid tumors or inflammation.

Pre-targeting refers to a step in a targeting method, wherein a primary target (e.g. a cell surface) is provided with a pre-targeting probe. The latter comprises a secondary target, which will eventually be targeted by a further probe equipped with a secondary targeting moiety.

Thus, in pre-targeting, a pre-targeting probe is bound to a primary target. The pre-targeting probe also carries secondary targets, which facilitate specific conjugation to a diagnostic (imaging) and/or therapeutic agent, the Effector Probe. After the construct forming the Pre-targeting Probe has localized at the target site (taking time, e.g. 24 to 72 h), a clearing agent may be used to remove excess from the blood, if natural clearance is not sufficient. In a second incubation step (preferably taking a shorter time, e.g., 1-6 hours), the effector probe binds to the (pre)bound pre-targeting probe via its secondary targeting moiety. The secondary target (present on the pre-targeting probe) and the secondary targeting moiety (present on the effector probe) should bind rapidly, with high specificity and high affinity and should be stable within the body.

Nanomedicines have demonstrated potential as targeting-vectors for diagnosis and/or therapy of cancer. Due to the leaky vasculature and reduced lymphatic drainage in some types of tumors in comparison to healthy tissue, long-circulating nano-sized agents tend to accumulate in tumors. This phenomenon is called the enhanced permeability and retention (EPR) effect. The EPR effect is a relevant approach for tumor targeting in pretargeted tumor imaging, where the targeting is separated from the actual imaging step. A pretargeted approach enables the use of short-lived radioisotopes, which reduces the radiation doses for the patients and increases imaging contrast.

Bio-orthogonal reactions are broadly useful tools with applications that span synthesis, materials science, chemical biology, diagnostics, and medicine. They are generally used in coupling reactions of small molecules, peptides, proteins, oligonucleotides, other types of polymers, glycans, nanoparticles, and on surfaces (e.g., glass slides, gold, resins). Further examples include: compound library synthesis, protein engineering, functional proteomics, activity-based protein profiling, target guided synthesis of enzyme inhibitors, chemical remodeling of cell surfaces, tracking of metabolite analogues, and imaging tagged.

A reference in this respect is WO 2010/051530, wherein pre-targeting is discussed on the basis of the reactivity between certain dienes, such as tetrazines and dienophiles such as a trans-cyclooctenol (TCO).

A pretargeted imaging approach may be based on a trans-cyclooctene (TCO) functionalized polyglutamic acid-graft-polysarcosine copolymer (PGA-graft-PSar-TCO) which allowed to accumulate within a tumor before a [¹¹¹In]In-labeled DOTA-tetrazine (Tz) derivative is administered intravenously (i.v.). The latter reacts with the TCOs on the polymer in an inverse electron demand Diels-Alder (iEDDA) reaction, which allows imaging of the polymer accumulation in the tumor.

### SUMMARY OF THE INVENTION

The present invention provides compositions based on a bio-orthogonal inverse electron demand Diels-Alder cycloaddition reaction for rapid and specific covalent attachment of a probe to a nanoparticle in vivo. The Diels-Alder reaction connects the two components of the reaction, a diene and a dienophile. The diene and dienophile are each physically connected, e.g., through a linker, either to a payload or to a nanoparticle. This bio-orthogonal chemistry platform can be used extracellularly or intracellularly, in vivo or in vitro. Thus, the invention includes using inverse electron demand Diels-Alder cycloaddition chemistry to chemically couple a diene with a dienophile to a polymeric nanoparticle.

The invention is based on polypept(o)idic graft copolymers that join a polypeptide backbone with poly(sarcosine) (polypeptoid) side chains. The polypeptide, e.g. polyglutamic acid, allows for covalent attachment of hydrophobic groups, such as reactive trans-cyclooctenes. Following cyclooctene attachment, polysarcosine chains can be attached for solubilizing and shielding purposes to form the final graft copolymer (comb copolymer) that coils in aqueous environment to spherical unimolecular nanoparticles with diameters between 8-20 nm.

Specifically, there is provided a polypept(o)idic comb (graft) copolymer for in vivo imaging by tetrazine transcyclooctene click chemistry, said comb (graft) copolymer having a polyglutamate backbone with transcyclooctene (TCO) bioorthogonal functional groups and polysarcosine chains covalently attached thereto; and wherein the comb polymer (graft) copolymer coils in aqueous environment to spherical nanoparticles with diameters between 5-30 nm; said copolymer is defined as:

p(Glu(COOH)ₙ-graft-(TCO)ₘ-graft-(pSar)ₖ)ₚ

wherein
Glu(COOH)ₙ denotes polyglutamate with n number of glutamate units, n ranging from 50 to 400;
(TCO)ₘ denotes transcyclooctene with m number of transcyclooctene unit ranging from substitution levels of pGlu form 5 to 40%;
(pSar)ₖ denotes polysarcosine with k number of polysarcosine units, k ranging from 20 to 200; and
   p denotes the number of pSar polymers in the comb (graft) copolymer; p ranging from 5 to 100 leading to a grafting density of the polysarcosine side chains of 2 to 50%.

Preferably, the polysarcosine is a homopolymer with degree of polymerization 60 to 100. In the above formula it is preferred that n ranges from 100 to 200, m ranges from substitution levels of pGlu form 10 to 30%, and/or p ranges from 10 to 50.

Concerning grafting density the polysarcosine side chains is preferably 5 to 40%, whereas the grafting density of TCO is preferably 1 to 40%.

Finally, it is preferred the spherical nanoparticles are unimolecular nanoparticle with a diameter of 8-20 nm.

In a second aspect of the present invention, there is provided a polypeptide-based carrier system comprising the polypept(o)idic comb (graft) copolymer defined above, and one or more tetrazine bioorthogonal functional groups each linked to a diagnostic agent.

The resulting nanoparticles are characterized by a high biocompatibility and pronounced. These systems were further shown to efficiently accumulate (10% injected dose per gram of tissue) in well-vascularized solid tumors after radioactive labeling by trans-cyclooctene tetrazine ligation (TCO-TZ ligation). Since the TCO-TZ ligation is bioorthogonal, the carrier system can also be labeled in vivo, which now makes it possible to image the polymers at any given time in the living organism. For the first time, this enables the detection of polymeric nanoparticles with short-lived radionuclides using SPECT or PET with the highest sensitivity and spatial resolution. This approach can be used, for example, for the diagnosis and therapy of solid tumors. Since passive accumulation of nanomedicines in solid tumors varies greatly between tumors and patients, patient selection is an important requirement for the clinical translation. Thus, a systems, which combines imaging of tumor accumulation with a radiotherapy using the same polymeric carrier, seems highly promising in cancer therapy and may improve long term survival of cancer patients.

These novel polymers are characterized by a high degree of tumor accumulation, a high amount of loading, and low synthesis costs. Surprisingly, these novel comb polymers enable accessibility of the highly hydrophobic TCO moiety on a water-soluble carrier system combined with its protection in the biological milieu and thus speed-up reaction kinetics of the TCO-TZ ligation more than 200 times, while in the case of TCOs attached to antibodies the rate constants decrease substantially.

The comb polymers can be used for the tetrazine ligation in vivo. This will result in better target-to-background ratios and consequently, to lower radiation burden in healthy tissue while maximizing radiotoxicity in the target region. As a result, the comb polymers can be used for personalized medicine to identify responders for nanoparticle-based drug delivery systems, such as Doxil oder Abraxane. Effectiveness of such treatment forms is strongly dependent on the EPR-effect, which is heterogeneous. Effective identification of the responders will allow wide-spread use of nanoparticle-based drug delivery systems and as such the market potential is huge. In addition, imaging and therapy can be combined in a step-wise protocol using tetrazine probes for imaging and radiotherapy. First, accumulation is imaged and whenever accumulation at the tumor side is pronounced the therapeutic probe can be applied. Therefore, imaging and therapy can be synergistically combined, which enhances the potential of the presented technology even further.

### BRIEF DESCRIPTION OF THE DRAWINGS

FIG. 1 shows nuclear imaging of polymers (A) Conventional imaging (B) Pretargeted imaging.

FIG. 2 displays the common structure of PGA-graft-PSar-TCOs.

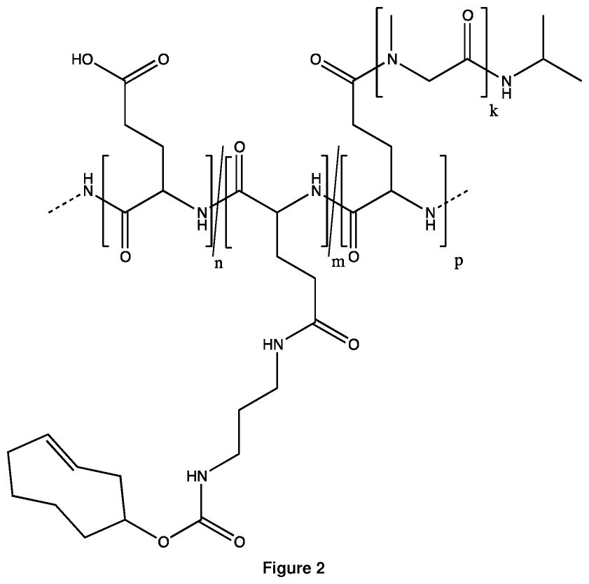

FIG. 3 displays the physical/chemical characterization of the synthesized PGA-graft-PSar-TCOs.

FIG. 4 shows the increased speed kinetic per TCO in our higher loading PGA-graft-PSar-TCOs.

### DETAILED DESCRIPTION OF THE INVENTION

The invention provides a platform technology to enable in vivo click chemistry for a combination of imaging and radiotherapy using the same polymeric carrier system.

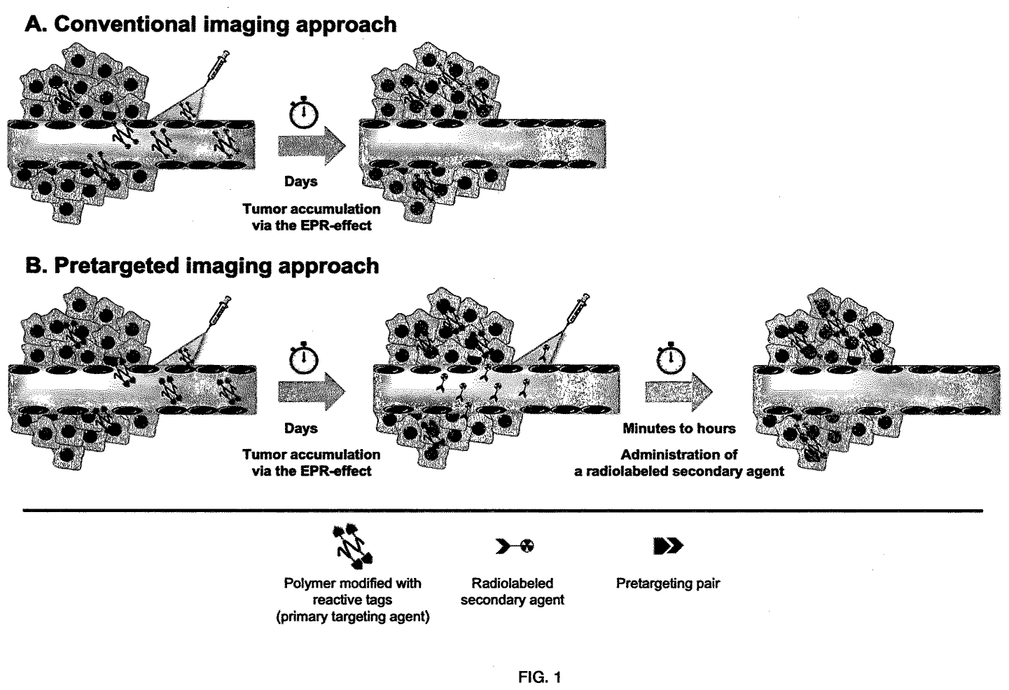

Traditionally, in conventional nuclear imaging a radionuclide is directly attached to the nano-agent before administration (FIG. 1). However, nano-sized agents often have slow pharmacokinetics, hence tumor accumulation generally takes several days and long-lived radionuclides are required to monitor their target accumulation. Consequently, the absorbed radiation doses for patients will be high and low imaging contrasts are obtained. These limitations can be circumvented by applying a pretargeted imaging approach, in which the nano-agent is first injected and allowed to accumulate to its target over a sufficient period of time, usually days, prior to the administration of a radiolabeled secondary imaging agent. The primary targeting agent, i.e. the nano-sized probe, and the secondary imaging agent are modified with compatible moieties, which will rapidly interact or react with each other in vivo. Thereby, any potential tumor accumulation of the nano-agent can be imaged. The reaction between the nano-agent and the secondary imaging agent on the tumor site can be achieved by the use of bioorthogonal chemistry. An outstanding bioothogonal reaction is the tetrazine ligation, performed between a 1,2,4,5-tetrazine (Tz) and a trans-cyclooctene (TCO). In addition to its bioorthogonality, this ligation shows high specificity and impressive reaction kinetics (rate constants up to 10⁶ M⁻¹ s⁻¹), criteria that make the tetrazine ligation optimal for pretargeting strategies. For pretargeting strategies based on the tetrazine ligation in nuclear imaging, the TCO-moieties are often attached to the primary targeting agent, whereas the Tz-framework is used as the secondary imaging agent. Most of the successful approaches for pretargeted tumor imaging have been using TCO-functionalized monoclonal antibodies (mAbs) in pair with Tz-derivatives radiolabeled with a number of different radionuclides. However, mAbs tend to be expensive and the modifications to conjugate the TCOs can be tedious. In addition, the amount of loading of hydrophobic groups, such as TCOs per mAb, is limited due to the risk of aggregation in the blood stream. Previously reported strategies with TCO-modified mAbs have most frequently used up to 3-11 TCOs/mAb.

In response to this, the present inventors developed a TCO-functionalized graft copolymer for use in pretargeted tumor imaging. In comparison to mAbs, polymers enable a high loading of TCOs/polymer without the risk of aggregation. In addition, and surprisingly, the rate constants per TCO/polymer increased using lipophilic tetrazines. Higher TCO loading increased the effect. This is an important finding since higher rate constants increase the likelihood/efficiency that pretargeted strategies occur in vivo. In the following a comprehensive experimental overview is provided to fully enable a skilled person to carry out the present invention.

### Subjects & Methods

The PGA-graft-PSar-TCO was synthesized by ring-opening polymerization of N-glutamic acid-O-tert-butyl ester (Glu(OtBu)) or N-glutamic acid-O-tert-benzyl ester (Glu(OBz)) carboxyanhydride, deprotection with TFA, postpolymerization modification with TCO (5 to 40%) and pSar (1 to 50%) by amide bond formation employing a coupling agent (e.g. DMTMM chloride). In the first step the polyglutamic acid backbone is synthesized by ring opening polymerization of Glu(OtBu) or (Glu(OBz)) carboxyanhydride using an amine initiator, e.g. neopentylamine. The degree of polymerization (DP) can be adjusted by the concentration of monomer divided by the concentration of initiator, which allows to set DP from 10-400. In the second step the protective group is cleaved by TFA or by a HBr/TFA mixture. The pSar side chains are synthesized in the same way employing Sarcosine N-carboxyanhydride. Afterwards, the polyglutamic acid is first modified with TCO using a coupling agent, e.g. DMTMM or HOBt/TBTU, and the polymer is purified by precipitation. Next the pSar chains are grafted onto the polymer using the same coupling agents. A slight access of pSar is required to achieve higher grafting densities. The final polymer is purified by dialysis, size exclusion chromatography or filtration to yield the pure graft copolymer.

The [¹¹¹In]In-DOTA-Tz was prepared as reported in the literature in a radiochemical conversion (RCC) of ≥99%. Analysis was performed by radio-TLC. In vivo stability studies and pretargeted microSPECT/CT imaging was performed using the ¹¹¹In-labeled PGA-graft-PSar, PGA-graft-PSar-TCO and [¹¹¹In]In-Tz-DOTA in BALB/c mice bearing subcutaneous colorectal mouse tumors (CT26). The reactivity of the PGA-graft-PSar-TCOs in the tetrazine ligation was determined by reaction with fluorogenic ‘turn-on’ Tz-derivatives HELIOS 347Me and HELIOS 388Me in a buffered aqueous environment.

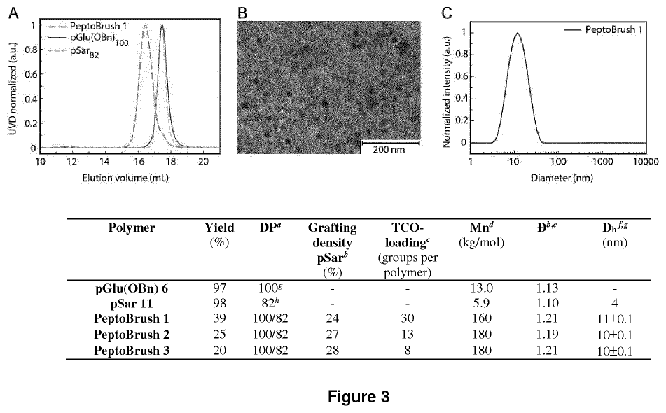

Results The PGA-graft-PSar-graft-TCO was labeled with [¹¹¹In]In-DOTA-Tz via the iEDDA reaction in phosphate buffer at room temperature for 5 min in a RCC of 82-85%. I.v. injection of ¹¹¹In-labeled PGA-graft-PSar resulted in tumor accumulation after 22 h. After establishing the stability and time for clearance of the polymer, the in vivo iEDDA reaction was tested by injecting PGA-graft-PSar-TCO i.v. 72 h before i.v. injection of the [¹¹¹In]In-DOTA-Tz. After 24 h a clear visualization of the tumor was observed. Moreover, a high loading of TCO-moieties (up to 30% side-chain functionalization) was achieved, without changing the physicochemical properties of the polymer. This high TCO load resulted in improved reaction kinetics up to 427 000 M⁻¹ s⁻¹. Interestingly, the observed rate constant could not solely be explained by the elevated number of TCO-moieties. A 29-fold increase was even observed when the rate constant was normalized to a single TCO-moiety (FIG. 3).

With respect to FIG. 3 it is to be noted that a) the degree of polymerization (DP) is the number of monomeric units in the polymer. Also, it should be noted that b) the determination by SEC in HFIP is relative to PMMA standards. c) Determined by 1H NMR in D2O or DMSO-d6. d) The number average molecular weight. e) The dispersity value (Ð) corresponds to the distribution of distinct molecular masses in a batch of polymers. f) Hydrodynamic diameter (Dh) of a polymer. g) Determined by dynamic laser light scattering at 173° in 10 mM NaCl solution. Values represent mean±standard of mean (S.E.M.) from n=3. g Determined by SEC in HFIP relative to PMMA and pSar standards. h) Determined by SEC in HFIP relative to PMMA and pSar standards.

### Conclusion

A new easily accessible PGA-graft-PSar copolymer functionalized with TCO for pretargeted tumor imaging based on the EPR effect and in vivo click chemistry has been developed. Its utility was validated with the previously reported [¹¹¹In]In-DOTA-Tz. Future work is to use this polymer in pretargeted tumor imaging in companion of Tz:s labeled with short-lived radioisotopes e.g. ¹¹C and ¹⁸F.

### Materials

Solvents and reagent were purchased from Sigma Aldrich and used as received unless otherwise noted. DMF was purchased from VWR, dried over molecular sieves (3 Å) and barium oxide and subsequently distilled in vacuo. Freshly distilled DMF was stored at −80° C. under exclusion of light. Prior to use, DMF was degassed under vacuum to remove residual dimethyl amine. THF and hexane were purchased from Sigma Aldrich and distilled from Na/K. Diethyl ether was distilled prior to use to remove the stabilizer. Other solvents were used as received. Milli-Q water (Millipore) with resistance of 18.2MΩ and TOC<3 ppm was used throughout the experiments. Diphosgene was purchased from Alfa Aesar and used as provided. Neopentylamine was purchased from TCl Europe, dried over sodium hydroxide and fractionally distilled. H-Glu(OtBu)-OH was purchased from Fluorochem, Hadfield, UK. Chloro-4,6-Dimethoxy-1,3,5-Triazin was obtained from Carbosynth, Compton, UK. [¹¹¹In]InCl₃ in hydrochloric acid was purchased from Mallinckrodt Medical B.V. Thin-layer chromatography (TLC) was carried out using either normal phase plates (silica gel 60 coated with fluorescent indicator F254s) or Reversed-phase modified silica plates (RP-18 modified silica gel 60 coated with fluorescent indicator F254s) from Merck. The fraction of radioactivity on the TLC-plates was measured with an instant imager from Packard. Analytical high performance liquid chromatography (HPLC) was performed on a Dionex system connected to a P680A pump, a UVD 170U detector and a Scansys radiodetector. Centrifugation was done in an eppendorf Centrifuge 5804R.

### Synthesis of Homopolymers

Sarcosine N-carboxyanhydride (NCA). Sarcosine NCA was synthesized as previously reported.¹⁷ Sarcosine (49.4 mg, 0.56 mol) was dried in vacuo for 2 hours and thereafter transferred to a flame-dried, 1 liter three-necked round bottom flask equipped with a stir bar, septum and reflux condenser, which was connected to two gas washing flasks filled with sodium hydroxide (35 g, x mol) in water (250 mL). Freshly THF (500 mL) were added under nitrogen counter-flow suspending the sarcosine. After ensuring a gas-tight apparatus by checking nitrogen leaving through the gas washing bottles, the nitrogen stream was turned off. Diphosgene (53.6 mL, 0.44 mol) were slowly added through the septum. After 2 hours at 65° C., additional diphosgene (5 mL, x mol) was added since the reaction was still turbid. After another hour at 65° C. a clear solution was effected and the septum was exchanged with a quick-fit, fitted with a glass tube through which a constant steam of nitrogen was bubbled though the solution for 3 hours, removing excess phosgene, hydrogen chloride and THF. The reaction was stored at −80° C. overnight. Remaining THF was removed in vacuo until a brown solid was obtained. This solid was re-suspended in THF (200 mL) and precipitated to dried and distilled hexane (1000 mL) in a sonic bath (obtaining 28 g crude product). The precipitate was collected and the hexane/THF mixture concentrated and precipitated again obtaining another 20 g of crude product. The precipitates were washed with hexane, dried in a constant stream of nitrogen. The precipitates were sublimated in 10 g batches at 1×10⁻³ bar at 85° C. Total yield after sublimation was (35 g, 55%). ¹H-NMR (400 MHz, DMSO-d₆): δ [ppm]=4.22 (2 H, s, CH₂—CO), 2.86 (3H, s, N—CH₃). Melting Point: 101.2-102.8° C. (Batch to batch variation, 2° C./min, starting at 85° C.).

γ-Tert-butyl-l-glutamic acid N-carboxyanhydride. The synthesis of γ-tert-butyl-1-glutamic acid (Glu(OtBu)) NCA was slightly optimized based on existing procedures.²¹ H-Glu(OtBu)-OH (15.0 g, 73.8 mmol) were thoroughly pestled and dried for 1 h under vacuum in a septum-sealed round bottom flask equipped with a stir-bar. Freshly distilled dried THF (300 mL) were used to suspend the solid. Freshly distilled triethylamine (20.4 mL, 148 mmol) was added, followed by addition of trimethylsilyl chloride (18.8 mL, 148 mmol). The suspension was stirred for 2 h at room temperature. To remove the precipitated salts, the solution was transferred in a nitrogen gas atmosphere and passed through a ceramic filter (16-40 μm pore size) into a three-necked round-bottomed flask equipped with a stir bar, a condenser, a rubber septum and a glass stopper. The filter residue was extracted twice with THF (50 mL) and extracts were added to the solution. Two gas washing bottles were connected to the condenser outlet and equipped with NaOH (8.86 g (3eq)) dissolved in water (250 mL). After verifying a gas-tight apparatus by observable bubbling in the gas washing bottles due to the nitrogen stream, the nitrogen stream was stopped after purging for an hour. Diphosgene (7.1 mL, 59 mmol) was added to the mixture at room temperature and heated to 50° C. during 1 h and to 70° C. for 30 min. The reaction was cooled to room temperature under a constant stream of nitrogen introduced via the septum. After room temperature was reached the septum was exchanged to a quick-fit and a stream of nitrogen was bubbled through a glass rod into the solution for 3 h. The flask was stored overnight at −80° C. and the residual solvent removed under high vacuum starting at low temperature. The solid residue was redissolved in THF (50 mL) and toluene (50 mL). The solution was precipitated to hexane (600 mL) under instant sonication, the white precipitate was collected by filtration under nitrogen atmosphere and dried under vacuum. (12.0 g, 71% yield) of fine white powder were obtained and recrystallized twice. ¹H-NMR (400 MHz, DMSO-d₆): δ [ppm]=9.07 (s, 1H, CO—NH—CHR), 4.45 (1H, ddd, J_{H,H}=7.6 Hz, 5.6 Hz, 1.2 Hz, NH—CH—CO), 2.34 (m, 2H, CH₂—CH₂—CO), 1.80-2.05 (m, 2H, CH₂—CH₂—CO), 1.40 (s, 9H, —O—C(CH₃)₃). Melting point: 102.5° C. (measured at 2° C./min).

Polysarcosine. Polysarcosine (pSar) was synthesized in a similar fashion as previously described, minor adjustments due to the volatile nature of the initiator were necessary.¹⁷ Sar-NCA (714 mg, 11.0 mmol) were transferred under nitrogen counter flow into a pre-dried Schlenk-tube equipped with a stir-bar and again dried under high vacuum for 1 h prior to reaction. The NCA was dissolved abs. DMF (10 mL) and 93.7 μL of a solution of 0.2 mL isopropylamine in 1.8 mL DMF were added for initiation with an Eppendorf pipette against nitrogen counter-flow. The solution was stirred overnight at room temperature and kept at a constant pressure of 1.25 bar of dry nitrogen via the Schlenk-line. Completion of the reaction was confirmed by FTIR spectroscopy (disappearance of the NCA peaks (1853 and 1786 cm⁻¹)). The polymer was precipitated into ether and centrifuged (4000 rpm at 4° C. for 10 min). After discarding the liquid fraction, new ether was added and the polymer was re-suspended in a sonic bath. The suspension was centrifuged again and the procedure was repeated. After DMF removal by the re-suspension steps, the polymer was dissolved in water and lyophilized, obtaining a fluffy polymer (436 mg, 98%). ¹H-NMR: (400 MHz, DMSO-d₆): δ [ppm]=4.70-3.70 (194H (2n), br, —NCH₃—CH₂—CO—), 3.10-2.60 (317H (3n), br, —NCH₃), 1.30-1.20 (1H, s —CH(CH₃)₂). 1.10-1.00 (6H, m, —CH(CH₃)₂). HFIP-SEC (vs PMMA Standards): Mₙ=22.4 kg/mol Ð=1.09, Degree of polymerization (DP) was determined to be 82 by calibration of apparent Mₙ against a series of pSar standards characterized by static light scattering to obtain absolute molecular weights (Weber et al.).

Poly(γ-tert-butyl-l-glutamic acid). Glu(OtBu)-NCA (475 mg, 2.07 mmol) were transferred under nitrogen counter flow into a pre-dried Schlenk-tube equipped with a stir-bar and dried under high vacuum for 1 h prior to solvation in a 1:1 mixture of abs. THF/abs. DMF (4 ml). A solution of neopentylamine (1.82 μL) in dry DMF (1.5 mL) was flushed with argon, before 1 mL of this solution was added to the Glu(OtBu)-NCA for initiation of polymerization. The mixture was stirred at 1° C. in order to prevent pyroglutamate termination, which can be present in glutamic acid polymerizations²² and kept at a constant pressure of 1.25 bar of dry nitrogen. Completion of the reaction was confirmed by FTIR spectroscopy (disappearance of the NCA peaks (1853 and 1786 cm⁻¹)). The polymer was precipitated into a cold mixture of ether/hexane 1:1 and centrifuged (4500 rpm at 4° C. for 15 min). After discarding the liquid fraction, new ether/hexane was added and the polymer was re-suspended in a sonic bath. The suspension was centrifuged again and the procedure was repeated. After DMF removal by the re-suspension steps, the polymer was dispersed in water and lyophilized, obtaining fine polymer flakes (272 mg, 69%). ¹H-NMR: (400 MHz, CDCl₃): δ [ppm]=8.80-7.80 (6.5H, br, —NH—CO—CH—), 7.54-6.98 (112H (5n), br, —C₆H₅), 5.30-4.65 (44H (2n), br, —O—CH₂—C₆H₅), 4.35-3.60 (0.78H (1n), br, —CO—CH—NH), 2.75-1.70 (3.46H, m, —CH₂—CH₂—), 1.65-1.20 (9H, s+br, O—C(CH₃)₃), 0.86 (0.66H, br CH₂—C(CH₃)₃ and grease). HFIP SEC (vs PMMA Standards): Mₙ=58.1 kg/mol, Ð=1.07.

Deprotection of poly(γ-tert-butyl-l-glutamic acid). pGlu(OtBu)₁₁₆ (100 mg) were dissolved in a 90:5:5 mixture of TFA:TIPS:MP-water (2 mL). The mixture was stirred at room temperature for 3 h and thereafter precipitated into ether. The precipitate was dissolved in MP-H₂O, bubbled with a steam of nitrogen to remove remaining ether, dialyzed against NaHCO₃ and lyophilized to afford deprotected p(Glu₁₁₆) (80 mg, 98%) as a fluffy polymer mass. ¹H-NMR: (400 MHz, D₂O): δ [ppm]=4.30-4.10 (116H (n), s, br —HN—CH—CO—), 2.30 (231H (2n), s, br, —CH₂—COOH), 2.05-1.70 (230H (2n), d, br CH—CH₂—CH₂—), 0.78 (9H, s, —C(CH₃)₃).

### TCO-Functionalization

4-(4,6-Dimethoxy-1,3,5-triazin-2-yl)-4-methyl morpholinium chloride. 4-(4,6-Dimethoxy-1,3,5-triazin-2-yl)-4-methyl morpholinium chloride (DMTMM Cl) was freshly prepared according to the literature.²³ N-methyl morpholine (1.73 g, 17.1 mmol) was added to a solution of 2-chloro-4,6-dimethoxy-1,3,5-triazine (3.00 g, 17.1 mmol) in dry THF (100 mL). The mixture was stirred at room temperature under nitrogen atmosphere for 1 h. The precipitate was collected by filtration under nitrogen atmosphere. After 2 hours of constant nitrogen flow through the precipitate and 2 hours under high vacuum DMTMM Cl (3.44 g, 73%) was afforded as colorless crystals, which were aliquoted into 2 mL-Eppendorf vials and stored at −20° C. ¹H-NMR: (400 MHz, DMSO-d₆): δ [ppm]=4.60-4.50 (2H, d, br, —N⁺CH₂—), 4.10-4.00 (8H, m, —OCH₃ and CH₃N⁺CH₂), 3.65-3.90 (4H, m, —OCH₂—), 3.45 (3H, s, —N⁺CH₃).

P(Glu(COOH)ₙ-ran-Glu(TCO)ₘ). The deprotected and lyophilized p(Glu₁₁₆) (40 mg, 0.286 mmol COOH), ((E)-cyclooct-4-en-1-yl(3-aminopropyl)carbamate) (16.5 mg, 0.0628 mmol, 0.21 eq) and NaHCO₃ (120 mg, 1.42 mmol, 5 eq) were dissolved in MP-water (4 mL) and DMSO (0.8 mL). The mixture was stirred at room temperature for 30 min, before After freshly prepared DMTMM Cl salt (79 mg, 0.29 mmol, 1eq) was added and the solution was stirred at room temperature under nitrogen atmosphere for 24 h. After 24 h additional DMTMM C (79 mg, 0.29 mmol, 1eq) was added and again the mixture was stirred for 24 h. The adduct was purified by dialysis against a 6-8 kDa molecular weight cut-off (MWCO) regenerated cellulose membrane for 1 week with daily change of water. After lyophilisation the TCO-functionalized polymer (34 mg, 62%) was obtained as a fluffy powder. ¹H-NMR: (400 MHz, D₂O): δ [ppm]=5.80-5.20 (46.7H (2m), m CH═CH) 4.40-3.80 (105H (m+n), —HN—CH—CO— and O—CH—(CH₂)₂), 3.75-3.60 (5H, m), 3.45-3.30 (5H, m), 3.20-2.90 (79H (˜4m) d, br), 2.70-2.50 (26H, s, br) 2.50-2.10 (244H (2n+2m), s, br, CH—CH₂—CH₂—), 2.10-1.65 (263H (2n+2m), d, br CH—CH₂—CH₂—), 1.65-1.35 (101H (˜4m), s, br CH₂—CH₂—CH₂), 0.76 (9H, s, br —C(CH₃)₃).

### Polysarcosinylation

p(Glu(COOH)ₙ-ran-Glu(TCO)ₘ-ran-Glu(pSar₈₂)ₖ). To p(Glu(COOH)ₙ-r-Glu(TCO)ₘ) (11.5 mg, 0.0667 mmol, 1eq Glu) was added pSar₈₂ homopolymer (200.5 mg, 0.0338 mmol, 0.5 eq (per Glu)) and NaHCO₃ (55 mg, 0,654 mmol, 10 eq). The reagents were dissolved in MP-water (4 mL) and DMSO (0.8 mL) and stirred at room temperature for 30 min. DMTMM C (37 mg, 0.134 mmol, 2 eq) was added and the mixture was stirred overnight. After 24 h, fresh DMTMM C (37 mg, 0.134 mmol, 2 eq) was added twice and SEC analysis (sampling of reaction solutions (3 μL) added to toluene (10 μL) in of HFIP (300 μL), filtered through a 450 nm PTFE filter as sample preparation) reveals increasing brush size after the first but not the second successive step, indicating close to saturated functionalization. The solution was then transferred into Centriprep™ centrifugation filters with a molecular weight cut-off of 30 kDa, diluted to a total volume of 15 mL with MP-water and spun 2×20 minutes. The filtrates were removed after every centrifugation step. After concentration, the filters were again diluted to a total volume of 15 mL with MP-water and centrifuged as previously described. The procedure was repeated 6 times until SEC analysis revealed no significant amounts of remaining pSar homopolymers. After lyophilisation the purified brush polymer (50 mg, 44%) was afforded. ¹H-NMR: (400 MHz, D₂O): δ [ppm]=5.70-5.30 (21H (2m), m, CH═CH), 4.70-3.80 (3400H (1n+1m+164k), HN—CH₂—CO+HN—CH—CO+O—CH—(CH₂)₂), 3.30-2.60 (5267H (246k), m N—CH₃), 2.70-2.50 (25H, s, br), 2.50-2.10 (54H (2n+2m+2k), s, br, CO—CH₂—), 2.10-1.65 (81H (2n+2m+2k), d, br CH—CH₂—CH₂, 1.65-1.35 (43H (˜4m), s, br), 1.10-1.00 (100H (6k), m, —CH(CH₃)₂). 0.78 (9H, s, —C(CH₃)₃).

### Kinetic Studies

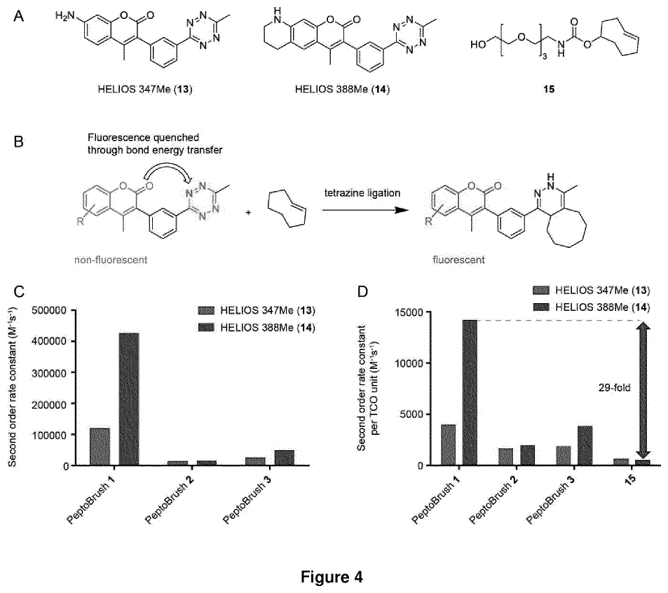

Reaction kinetics of the TCO-moieties in PGA-graft-PSar-TCOs and TCO with fluorogenic turn-on Tz-derivatives HELIOS 347Me and HELIOS 388Me were determined by pseudo-first order measurements in PBS (pH=7.4) at 37.0±0.1° C. following the increase of fluorescence at >400 nm. Measurements were performed using a SX20 stopped flow photometer (Applied Photophysics, UK) equipped with a 360 nm LED light source and a photomultiplier type R374 in combination with a 400 nm longpass filter as detector. 20 mM stock solutions of HELIOS 347Me and HELIOS 388Me in DMSO-d6 were diluted in PBS (1:40000) resulting in 500 nM solutions. Solutions of PGA-graft-PSar-TCOs were prepared to yield TCO concentrations exceeding 10 μM to ensure pseudo first order conditions. Tz and TCO solutions were mixed 1:1 during measurements, resulting in 250 nM solution of HELIOS 347Me and HELIOS 388Me and TCO concentrations ≥5 μM. The used concentrations observed rate constants and calculated second order rate constants are shown in FIG. 4.

### ¹¹¹In-Labeling of DOTA-Tz.

To a solution of 1 M NH4OAc pH 5.0 (10 μL) and DOTA-Tz precursor (0.1 mg, 78 nmol, 50 μL from stock solution in 0.2 M NH₄OAc pH 7.0) was added [¹¹¹In]InCl₃ (˜1 mL, 372-647 MBq). The mixture was heated at 60° C. for 5 min, before 13 mM gentisic acid in saline (29.3 μL) and 10 mM DTPA in PBS (5 μL) were added. The mixture was heated at 60° C. for 5 additional min. Analysis was performed by radio-TLC with 200 mM EDTA in MQ-water as eluent and with radio-HPLC on a Yarra™ 3 μm SEC-2000 LC column (300×7.8 mm) with Na₂HPO₄/NaH₂PO₄ buffer (pH 7.0) as eluent. [¹¹¹In]In-DOTA-Tz was afforded in a RCY of 99% and a RCP of >97% (see radio-TLC and radio-HPLC chromatogram in supporting information).

### ¹¹¹In-Labeling of TCO-pGlu-Graft-pSar

DOTA-Tz was labeled with ¹¹¹In as described above. The reaction mixture was diluted with PBS (0.7-1 mL). Thereafter TCO-pGlu-graft-pSar (3.4 mg) dissolved in PBS (0.3 mL) was added. After 10 min at room temperature, the reaction was analysed by radio-HPLC on a Yarra™ 3 μm SEC-2000 LC column (300×7.8 mm) with Na₂HPO₄/NaH₂PO₄ buffer (pH 7.0) as eluent. [¹¹¹In]In-pGlu-graft-pSar was afforded in a 99% RCY and with a RCP of >96%.

1. A polypept(o)idic comb (graft) copolymer for in vivo imaging by tetrazine transcyclooctene click chemistry, said comb (graft) copolymer having a polyglutamate (pGlu) backbone with transcyclooctene (TCO) bioorthogonal functional groups and polysarcosine chains covalently attached thereto; and wherein the comb polymer (graft) copolymer coils in aqueous environment to spherical nanoparticles with diameters between 5-30 nm; said copolymer is defined as:
p(G\Uₙ-graft-(TCO)ₘ-graft-(pSar)ₖ)ₚ
wherein
Glun denotes polyglutamate with n number of glutamate units, n ranging from 50 to 400;
(TCO)ₘ denotes transcyclooctene with m number of transcyclooctene unit ranging from substitution levels of pGlu form 5 to 40%;
(pSar)ₖ denotes polysarcosine with k number of polysarcosine units, k ranging from 20 to 200; and
p denotes the number of pSar polymers in the comb (graft) copolymer; p ranging from 5 to 100 leading to a grafting density of the polysarcosine side chains of 2 to 50%.

2. The polypept(o)idic comb (graft) copolymer of claim 1, wherein polysarcosine is a homopolymer with degree of polymerization 60 to 100.

3. The polypept(o)idic comb (graft) copolymer of claim 1, wherein n ranges from 100 to 200.

4. The polypept(o)idic comb (graft) copolymer of claim 1, wherein m ranges from substitution levels of pGlu form 10 to 30%.

5. The polypept(o)idic comb (graft) copolymer claim 1, wherein p ranges from 10 to 50.

6. The polypept(o)idic comb (graft) copolymer of claim 1, wherein the grafting density of the polysarcosine side chains is 5 to 40%.

7. The polypept(o)idic comb (graft) copolymer of claim 1, wherein the grafting density of TCO is 1 to 40%.

8. The polypept(o)idic comb (graft) copolymer of claim 1, wherein the spherical unimolecular nanoparticle has a diameter between 8-20 nm.

9. A polypeptide-based carrier system comprising:
the polypept(o)idic comb (graft) copolymer of claim 1, and
one or more tetrazine bioorthogonal functional groups each linked to a imaging agent or a therapeutic agent.

10. A method for tumour imaging, the method comprising administering a polypept(o)idic comb (graft) copolymer according to claim 1 to a subject and allowing to circulate in the subject's system for a period of time effective to achieve binding to a tumour target, followed by clearing non-bound copolymer from the body, followed by administering one or more tetrazine bioorthogonal functional groups each linked to an imaging agent.

11. A method for tumour treatment, the method comprising administering a polypept(o)idic comb (graft) copolymer according to claim 1 to a subject and allowing to circulate in the subject's system for a period of time effective to achieve binding to a tumour target, followed by clearing non-bound copolymer from the body, followed by administering one or more tetrazine bioorthogonal functional groups each linked to an therapeutic agent.
